(19) United States
(12) Patent Application Publication
(10) Pub. No.: US 2007/0237786 A1
(43) Pub. Date: Oct. 11, 2007

(54) Diet supplement for causing rapid weight loss, controlling appetite, managing stress, supporting relaxation, combating fatigue and supporting mental well-being.

(75) Inventor: Marvin A. Heuer, Mississauga (CA)

Correspondence Address: IOVATE HEALTH SCIENCE RESEARCH INC., 5100 SPECTRUM WAY, MISSUSSAUGA, ON, L4W 5S2, CA

(73) Assignee: Smartburn Formulations Ltd, Mississauga (CA)

(21) Appl. No.: 11/765,476

(22) Filed: Jun. 20, 2007

Related U.S. Application Data
(62) Division of application No. 11/453,159, filed on Jun. 13, 2006.
(60) Provisional application No. 60/691,945, filed on Jun. 17, 2005.

(51) Int. Cl. A61K 36/00; A61K 36/02; A61K 36/18; A61P 3/04

(52) U.S. Cl. 424/195.17; 424/725

(57) ABSTRACT

Compositions and methods for administering to the diet of humans a composition for inducing rapid weight loss, controlling appetite, managing stress and supporting mental well-being, supporting relaxation, and combating fatigue. A diet supplement comprising Calcium and Potassium double salt of Garcinia Cambogia Extract supplying 60% Hydroxycitric Acid, Gymnema Sylvestre Leaf Extract, Rhodiola Rosea Root Extract, Theanine Astaxanthin Algae Extract, Chromium Polynicotinate, Hoodia Gordonii, N-olyl-phosphatidyl ethanolamine (NOPE)/EGCG blend, Vinpocetine, Russian Tarragon Extract, N-acetyl tyrosine, and Withania Somnifera Root Extract is provided. Said diet supplement is comprised of at least Hoodia Gordonii wherein the extract does not contain any extract from the root of the plant.

### RELATED APPLICATIONS

The application is related to and claims benefit of priority to the Applicant's co-pending U.S. Provisional Patent Application Ser. No. 60/691,945 entitled “Diet Supplement for Causing Rapid Weight Loss, Controlling Appetite, Managing Stress, Supporting Relaxation, Combating Fatigue and Supporting Mental Well-Being,” filed Jun. 17, 2006, the disclosure of which is hereby fully incorporated by reference.

### FIELD OF THE INVENTION

The present invention relates to a diet supplement for causing rapid weight loss, controlling appetite, managing stress, supporting relaxation, combating fatigue and supporting mental well-being. Preferably, the diet supplement is provided in a caplet form consumable several times per day, for causing rapid weight loss, controlling appetite, managing stress, supporting relaxation, combating fatigue and supporting mental well-being throughout the day. Additionally, the present invention relates to a method of promoting same by consuming the diet supplement. In addition, the present invention relates to a method of manufacturing the diet supplement.

### SUMMARY OF THE INVENTION

The present invention provides for a diet supplement causing rapid weight loss, controlling appetite, managing stress, supporting relaxation, combating fatigue and supporting mental well-being. The diet supplement may include one or more of the Calcium and Potassium double salt of Garcinia Cambogia Extract standardized to about 60% Hydroxycitric Acid, Gymnema Sylvestre Leaf Extract, Rhodioia Rosea Root Extract, Theanine (gamma-glutamylethylamide). Astaxanthin, Chromium Polynicotinate, Hoodia Gordonii (e.g., an extract that does not contain any extract from the root of the plant), N-olyl-phosphatidyl ethanolamine (NOPE)/EGCG blend, Vinpocetine, Russian Tarragon Extract (Artemisia dracunculus L. plant). N-acetyl tyrosine and Withania Somnifera Root Extract. For example, in an embodiment, the present invention may provide a diet supplement comprising an extract of Hoodia Gordonii and Vinpocetine, wherein the Hoodia Gordonii extract does not contain any extract from the roots of the plant. The diet supplement may also include any one or more of an extract of Rhodiola Rosea, Theanine, an Astaxanthin extract of Algae, Chromium Polynicotinate, a blend of N-olyl-phosphatidyl ethanolamine/EGCG Blend, an extract of Russian Tarragon, N-acetyl tyrosine, an extract of Withania Somnifera, an extract of Gymnema Sylvestre, and the Calcium and Potassium double salt of Garcinia Cambogia Extract supplying about 60% Hydroxycitric Acid.

Advantageously, the diet supplement is provided in a caplet form, which may be consumed several times per day. For instance, in an embodiment, the diet supplement comprises a two caplet serving; each serving suitable for being consumed three times daily, e.g., before each one of three daily meals. In this manner, the diet supplement may cause rapid weight loss, control appetite, manage stress, support relaxation, combat fatigue and support mental well-being for an extended period of time, e.g., all day.

The present invention also provides, by the consumption of the supplemental composition, a method for causing rapid weight loss, controlling appetite, managing stress, supporting relaxation, and supporting mental well-being. For example, in an embodiment, the present invention may provide a method for controlling appetite and promoting rapid weight loss, comprising the step of administering to a human or animal a diet supplement that comprises an extract of Hoodia Gordonii, wherein the Hoodia Gordonii extract does not contain any extract from the roots of the plant, and may also comprise one or more of the Calcium and Potassium double salt of Garcinia Cambogia Extract supplying 60% Hydroxycitric Acid. Gymnema Sylvestre, Chromium Polynicotinate, N-olyl-phosphatidyl Ethanolamine/EGCG Blend, an extract of Russian Tarragon and Vinpocetine. The method may include the step of administering the diet supplement to a human or animal at least once daily.

In addition, the present invention relates to a method of manufacturing a diet supplement for causing rapid weight loss, controlling appetite, managing stress, supporting relaxation, combating fatigue and supporting mental well-being. In an embodiment, there is provided a method of manufacturing a diet supplement comprising one or more of the Calcium and Potassium double salt of Garcinia Cambogia Extract standardized to about 60% Hydroxycitric Acid, Gymnema Sylvestre Leaf Extract, Rhodiola Rosea Root Extract, Theanine (gamma-glutamyethylamide), Astaxanthin, Chromium Polynicotinate, Hoodia Gordonii (e.g., an extract that does not contain any extract from the root of the plant), N-olyl-phosphatidyl ethanolamine (NOPE/EGCG blend, Vinpocetine, Russian Tarragon Extract (Artemisia dracunculus L. plant), N-acetyl tyrosine and Withania Somnifera Root Extract.

In addition, the present invention may relate to a method of managing stress comprising the step of administering to a human or animal a diet supplement that comprises an extract of Rhodiola Rosea, Theanine, N-acetyl Tyrosine and an extract of Withania Somnifera Root. In addition, the present invention may relate to a method of supporting relaxation comprising the step of administering to a human or animal a diet supplement that comprises Theanine and an extract of Russian Tarragon. In addition, the present invention may relate to a method of supporting mental well-being comprising the step of administering to a human or animal a diet supplement that comprises an extract of Rhodiola Rosea, Theanine, Astaxanthin extract of Algae, Vinpocetine, N-acetyl Tyrosine and an extract of Withania Somnifera Root. In addition, the present invention may relate to a method of ameliorating mental performance comprising the step of administering to a human or animal a diet supplement that comprises Vinpocetine, an extract of Rhodiola Rosea, and N-acetyl Tyrosine. In addition, the present invention may relate to a method of regulating blood glucose levels comprising the step of administering to a human or animal a diet supplement that comprises an extract of Gymnema Sylvestre, Chromium Polynicotinate, and an extract of Russian Tarragon. In addition, the present invention may relate to a method of supplying antioxidants to a human or animal comprising the step of administering to a human or animal a diet supplement that comprises an extract of Rhodiola Rosea, Theanine, an extract of Astaxanthin, N-olyl-phosphatidyl Ethanolamine/EGCG Blend, and an extract of Withania Somnifera Root.

### DETAILED DESCRIPTION OF THE INVENTION

The present invention, according to various embodiments thereof, is directed to a diet supplement for inducing rapid weight loss, controlling appetite, and managing stress, supporting relaxation, combating fatigue and supporting mental well-being.

Hoodia Gordonii (wherein the extract does not contain any extract from the root of the plant).

Hoodia is a cactus which has been used traditionally to ease hunger discomfort and as such, is used as an appetite suppressant. A suspected active compound isolated from Hoodia Root, termed P57, has been shown to reduce food intake in rats (MacLean D B. Luo L G. Increased ATP content/production in the hypothalamus may be a signal for energy-sensing of satiety, studies of the anorectic mechanism of a plant steroidal glycoside. Brain Res. 2004 Sep 10; 1020(1-2):1-11.). Furthermore, it was found that P57 countered the reduction in ATP normally expected from calorie restriction. Further studies in rats indicate that Hoodia is effective in reducing blood glucose and weight loss (Tulp O L, Harbl N A, Mihalov J, DerMarderosian A. Effect of Hoodia plant on food intake and body weight in lean and obese LA/Ntul//cp rats. FASEB J. 2001 Mar 7, 15(4):A404.: Tulp O L Harbi N A, DerMarderosian A. Effect of Hoodia plant on weight loss in congenic obese LA/Ntul//-cp rats FASEB J. 2002 Mar 20:16(4):A648.).

In 2001, Phytopharm completed a double-blind, placebo-controlled trial comprised of a group of health yet overweight volunteers. Following two weeks of high doses of Hoodia, the treatment group showed a reaction in weight while being inactive, and further, their daily caloric intake was voluntarily reduced via appetite suppression by approximately 1000 calories.

In an embodiment of the present invention, which is set forth in greater detail in Example 1, the diet supplement may include Hoodia Gordonii (e.g., an extract that does not contain any extract from the root of the plant). A serving of the diet supplement may include from about 1.0 mg to about 100 mg of Hoodia Gordonii (e.g., an extract that does not contain any extract from the root of the plant). The preferred dosage, in a serving of said diet supplement, comprises about 20 mg of Hoodia Gordonii (e.g., an extract that does not contain any extract from the root of the plant).

Vinpocetine

Vinpocetine is an extract of Vinca minor, or periwinkle plant, used traditionally to improve circulation. Vinpocetine has been shown to prevent brain cell damage by increasing blood flow in a rat model of forebrain ischemia (2-vessel occlusion and hypotension) wherein the ischemia was maintained for 10 minutes (Sauer D, Rischke R, Beck T, Rossberg C, Mennel H D, Bielenberg G W, Kriegistein J, Vinpocetine prevents ischemic cell damage in rat hippocampus. Life Sci. 1998: 43(21).17333-9). Image analysis indicates that Vinpocetine can increase cerebral blood flow in stroke patients and thereby improving cerebral glucose uptake and metabolism in the brain (Szakail S, Boros I, Balkay L, Emri M, Fekete I, Kerenyi L, Lehel S, Marian T, Molnar T, Varga J, Galuska L, Tron L, Bereczki D, Csiba L, Gulyas B, Cerebral effects of a single dose of intravenous vinpocetine in chronic stroke patients; a PET study. J Neuroimaging. 1998 Oct;8(4):197-204: Szilagyi G, Nagy Z, Balkay L, Boros I, Emri M, Lehel S, Marian T, Moinar T, Szakail S, Tron L, Bereczki D, Csiba L, Fekete I, Kerenyi L, Galuska L, Varga J, Bonoczk P, Vas A, Gulyas B. Effects of vinpocetine on the redistribution of cerebral blood flow and glucose matabolism in chronic ischemic stroke patients; a PET study. J Neurol Sci 2005 Mar 15:229-230:275-84). Interestingly, Vinpocetine may exert its effect by blocking sodium channels in the brain (Moinar P, Erdo S L. Vinpocetine is as potent as phenyloin to block voltage-gated Na+ channels in rat cortical neurons. Eur J Pharmacol 1995 Feb 6:273(3):303-6), which is thought to be involved in neuroprotection (Bonoczk P, Gulyas B, Adam-Vizi V, Nemes A, Karpali E, Kiss B, Kapas M, Szantay C, Koncz I, Zelles T, Vas A, Role of sodium channel inhibition in neuroprotection effect of vinpocetine. Brain Res Bull, 2000 Oct: 53(3):245-54). The antioxidant activity of Vinpocetine may also contribute to protection of neural cells (Santos M S, Duarte A I, Moreira P I, Oliveira C R, Synaptosomal response to oxidative stress effect of vinpocetine. Free Radic Res. 2000 Jan; 32(1):57-66.). Furthermore, Vinpocetine may also prevent the accumulation of calcium, phosphorus and aluminum in the central nervous system, which has been suggested to be involved in atherosclerosis (Yasui M, Yano I, Ota K, Oshima A, Calcium phosphorus and aluminum concentrations in the central nervous system, liver and kidney of rabbits with experimental atherosclerosis preventive effects of vinpocetine on the deposition of these elements, J Int Med Res 1990 Mar-Apr: 18(2):142-52.).

In human trials, Vinpocetine has been shown to be effective at treating a number of circulation-related disorders. In a double-blind clinical trial, Vinpocetine was shown to effect significant improvement in elderly patients with chronic cerebral dysfunction (Balestreri R, Fontana L, Astengo F. A double-blind placebo controlled evaluation of the safety and efficacy of vinpocetine in the treatment of patients with chronic vascular senile cerebral dysfuction. J Am Geriatr Soc 1987 May, 35(6):425-30). A single-blind randomized trial has demonstrated the feasibility of continued study of Vinpocetine to treat stroke patients (Feigin V L, Doronin B M, Popova T F, Gribatcheva E V, Tchervov D V. Vinpocetine treatment in acute ischaemic stroke; a pilot single-blind randomized clinical trial. Eur J Neurol 2001 Jan; 8(1):81-5.). Further to these studies, it was shown that cognitive performance improved in patients with mild to moderate pychosyndromes such as dementia (Hindmarch I, Fuchs H H, Erzigkeit H. Efficacy and tolerance of vinpocetine in ambulant patients suffering from mild to moderate organic psychosyndromes Int Clin Psychopharmacol. 1991 spring;6(1):31-43.). An improvement in the memory of healthy woman due to a three-day supplementation with Vinpocetine has also been documented (Subhan Z, Hindmarch I. Psychopharmacological effects of vinpocetine in normal healthy volunteers. Eur J Clin Pharmacol. 1985:28(5):567-71).

In an embodiment of the present invention which is set forth in greater detail in Example 1, include from about 0.1 mg to about 10 mg of Vinpocetine. The preferred dosage, in a serving of said diet supplement, comprises about 1 mg of Vinpocetine.

Theanine (Gamma-Glutamylethylamide)

Theanine is an amino acid found in green tea. It is however distinct from the polyphenols and Catechins that are typically associated with the beneficial effects of green tea. While Catechins are generally associated with antioxidant activity, Theanine is associated with anti-stress and cortisol control.

In hypertensive rats, Theanine has been shown to lower blood pressure (Yokogoshi H, Kato Y, Sagesaka Y M, Takinara-Matsuura T, Kakuda T, Takeuchi N. Reduction effect of theanine on blood pressure and brain 5-hydroxyindoles in spontaneously hypertensive rats. Biosci Biotechnol biochem. 1995 Apr;59(4):615-8). Moreover, Theanine possess neuroprotective effects in animal models of brain damage (Kakuda T, Yanase H, Utsunomlya K, Nozawa A, Unno T, Kataoka K. Protective effect of gamma-glutamylethylamide (Theanine) on ischemic delayed neuronal death in gerbils. Neurosci Lett. 2000 Aug 11;289(3):189-92.) which is thought to be due to inhibiting the ligand binding to glutamate receptors (Kakuda T, Nozawa A, Sugimoto A, Niino H. Inhibition by theanine of binding of [3H]AMPA, [3H]kainate, and [3H]MDL 105,519 glutamate receptors. Biosci Biotechnol Biochem. 2002 Dec:66(12):2683-6.). Glutamic acid signaling through its related glutamate receptor is involved in memory and learning. Excess glutamate receptor signaling, which may occur due to and following brain injury, can lead to neuronal cell death (Chol D W, Rothman S M. The role of glutamate neurotoxicity in hypoxic-ischemic neuronal death. Annu Rev Neurosci 1990:13:171-82.) via apoptotic cascade mechanisms. Furthermore, weight gain and fat accumulation have been suppressed in rats fed Theanine (Zheng G, Sayama K, Okubo T, Juneja L R, Oguni I, Anti-obesity effects of three major components of green tea, catechins, caffeine and theanine, in mice. In Vivo 2004 Jan-Feb:18(1):56-62.) relative to control animals.

Clinical studies have been done examining effects of Theanine in humans. One study demonstrated a relaxing effect under non-stress conditions (Lu K, Gray M A, Oliver C, Liley D T, Harrison B J, Bartholomeusz C F, Phan K L, Nathan P J. The acute effects of L-theanine in comparison with alprazolam on anticipatory anxiety in humans. Hum Psychopharmacol, 2004 Oct;19(7):457-65.).

In an embodiment of the present invention, which is set forth in greater detail in Example 1, the diet supplement may include Theanine (gamma-glutamylethylamide). A serving of the diet supplement may include from about 1.0 mg to about 100 mg of Theanine (gamma-glutamylethylamide). The preferred dosage, in a serving of said diet supplement, comprises about 52 mg of Theanine (gamma-glutamylethylamide).

Astaxanthin

Astaxanthin is a red carontenoid pigment occurring naturally in many living organisms. Studies utilizing animals indicate that Astaxanthin can confer antioxidant activity that can altenuate exercise-induced muscle damage (Aoi W, Naito Y, Sakuma K, Kuchide M, Tokuda H, Macka T, Toyokuni S, Oka S, Yasuhara M, Yoshikawa T. Astaxanthin limits exercise-induced skeletal and cardiac muscle damage in mice. Antioxid Redox Signal 2003 Feb:5(1):139-44), has anticancer activity (Jyonouchi H, Sun S, Lijima K, Gross M D. Antitumor activity of astaxanthin and its mode of action. Nutr Cancer. 2000:36(1):59-65). anti-inflammatory activity (Kurashige M, Okimasu E, Inoue M, Utsumi K. Inhibition of oxidative injury of biological membranes by astaxanthin. Physiol Chem Phys Med NMR 1990:22(1);27-38.). anti-diabetic activity (Uchiyama K, Naito Y, Hasegawa G, Nakamura N, Takahashi J, Yoshikawa T. Astaxanthin protects beta-cells against glucose toxicity in diabetic db/db mice. Redox Rep 2002:7(5):290-3.), immunity-boosting properties (Okai Y, Higashi-Okai K. Possible immunomodulating activities of carotenoids in vitro cell culture experiments Int J Immunopharmacol. 1996 Dec;18(12):753-8.), and antihypertensive and neuroprotective properties (Hussein G, Nakamura M, Zhao Q, Iguchi T, Goto H, Sankawa U, Watanabe H. Antihypertensive and neuroprotective effects of astaxanthin in experimental animals. Biol Pharm Bull. 2005 Jan;28(1):47-52.).

Studies have examined the effects of Astaxanthin in humans where it has been shown to be safe (Spiller G A, Dewell A. Safety of an astaxanthin-rich Haematococcus pluvialis algal extract, a randomized clinical trial. J Med Food 2003 spring;6(1);51-6.) and of potential benefit in cancer treatment by inhibiting 5-alpha-reductase (Anderson M L. A preliminary investigation of the enzymatic inhibition of 5alpha-reduction and growth of prostatic carcinoma cell line LNCap-FGC by natural astaxanthin and Saw Palmetto lipid extract in vitro. J Herb Pharmacother, 2005;5(1);17-26.).

In an embodiment of the present invention, which is set forth in greater detail in Example 1, the diet supplement may include Astaxanthin. A serving of the diet supplement may include from about 1 mg to about 100 mg of Astaxanthin. The preferred dosage, in a serving of said diet supplement, comprises from about 5 mg of Astaxanthin.

Chromium Polynicotinate

Chromium is an essential trace mineral that is used to control blood sugar levels by aiding insulin binding, wherein it can aid in the control weight of reduction. Chromium, however, is poorly absorbed by the body and must therefore be combined with a more efficiently absorbed compound such as niacin (found in Polynicotinate). Chromium likely exerts its main function as a component of the glucose tolerance factor, which is involved in insulin sensitivity.

Chromium has been shown clinically to increase lean mass (Bahadori B, Wailner S, Schneider H, Wascher T C, Toplak H, Effect of chromium yeast and chromium picolinate on body composition of obese, non-diabetic patients during and after a formula diet. Acta Med Austriaca, 1997:24(5):185-7.) and reduce body fat when combined with exercise (Grant K E, Chandler R M, Castle A L, Ivy J L, Chromium and exercise training; effect on obese women, Med Sci Sports Exerc. 1997 Aug:29(8:992-8.). Chromium has also been shown to increase HDL, (‘good’) cholesterol (Riales R, Albrink M J. Effect of chromium chloride supplementation on glucose tolerance and serum lipids including high-density lipoprotein of adult men Am J Clin Nutr 1981:34:2670-8.).

In an embodiment of the present invention which is set forth in greater detail in Example 1, the diet supplement may include Chromium Polynicotinate. A serving of the diet supplement may include from about 0.01 mg to about 10 mg of Chromium Polynicotinate. The preferred dosage, in a serving of said diet supplement, comprises about 0.133 meg (0.000133 g) of Chromium Polynicotinate.

N-olyl-Phosphatidyl Ethanolamine (“NOPE”)/EGCG Blend

NOPE is a naturally occuring lipid synthesized and released in the intestine, in vivo rat studies have shown that food deprivation results in decreased NOPE synthesis, while administration of NOPE causes appetite suppression and subsequent weight loss (Rodriguez de Fonseca F, Navarro M, Gomez R, Escuredo L, Nava F, Fu J, Munillo-Rodriguez E, Giuffrida A, LoVerme J, Gaetani S, Kathuria S, Gall C, Piomelli D. An anorexic lipid mediator regulated by feeding Nature 2001 Nov 8;414(6860):209012., Gaetani S, Oveisi F, Piomelli D. Modulation of meal pattern in the rat by the anorexic lipid mediator olecylethanolamide. Neuropsychopharmacology, 2003 Jul;28(70:1311-6.). Moreover, NOPE has been shown to lower body weight in obese rats (Fu J, Oveisi F, Gaetani S, Lin E, Piomelli D. Oleoylethanolamide, an endogenous PPAR-alpha agonist, lowers body weight and hyperlipidemia in obese rats. Neuropharmacology, 2005 Jun;48(8):1147-53.). This action is likely mediated through the NOPE binding of the peroxisome-proliferator-activated receptor-alpha (PPAR-alpha) (Fu J, Gaetani S, Oveisi F, Lo Verme J, Serrano A, Rodriguez De Fonseca F, Rosengarth A, Luecke H, Di Giacomo B, Tarzia G, Piomelli D. Oleylethanolamide regulates feeding and body weight through activation of the nuclear receptor PPAR-alpha Nature 2003 Sep 4;425(6953):90-3.). PPAR-alpha is involved in regulating lipid metabolism (Chawla A, Repa J J, Evans R M, Mangeisdorf D J. Nuclear receptors and lipid physiology: opening the X-files. Science 2001 Nov 30:294(5548):1866-70.). Furthermore, another possible mechanism of NOPE's action is through the 7-transmembrane spanning G protein-coupled receptor (GPCR) GPR119 found predominantly in human and rodent pancreas (Overton H A, Babbs A J, Doel S M, Fyfe M C, Gardner L S, Griffin G, Jackson H C, Procter M J, Rasamison C M, Tang-Christensen M, Widdowson P S, Williams G M, Reynet C, Deorphanization of a G protein-coupled receptor for oleoylethanoiamide and its use in the discovery of small-molecule hypophagic agents. Cell Metab. 2006 Mar;3(3):167-75.) by which hypophagic signals may be conferred.

(-)-Epigallocatechin-3-gallate (EGCG) is the most active Catechin polyphenol compound found in Green Tea. EGCG has potent antioxidant activity and has been shown by laboratory tests to be greater than many well known and established antioxidants such as vitamin C and E (Pillar S P, Mitscher L A, Menon S R, Pillal C A. Shankel D M. Antimutagenic/antioxidant activity of green tea components and related compounds. J Environ Pathol Toxicol Oncol. 1999:18(3):147-58). Further to its antioxidant activity, EGCG was found to be effective at reducing food intake, body weight, cholesterol and triglycerides in both lean and obese rats (Kao Y H, Hipakka R A, Liao S. Modulation of endocrine systems and food intake by green tea epigailocatechin gailate. Endocrinology,2000 Mar;141(3):980-7.). In humans, Green Tea extracts rich in EGCG and other Catechins have been shown to result in a rapid increase in plasma antioxidant activity (Benzie I F, Szeto Y T, Strain J J, Tomlinson B. Consumption of green tea causes rapid increase in plasma antioxidant power in humans, Nutr Cancer 1999:34(1):83-7.) and further to aid in weight loss due to increased metabolism and fat oxidation (Chantre P, Lairon D. Recent findings of green tea extract AR25 (Exolise) and its activity for the treatment of obesity. Phytomedicine 2002 Jan;9(1):3-8. Dulico A G, Duret C, Rohrer D, Girardier L, Mensi N, Fathi M, Chantre P, Vandermander J. Efficacy of a green tea extract rich in catachin polyphenols and caffeine in increasing 24-h energy expenditure and fat oxidation in humans. Am J Clin Nutr. 1999 Dec;70(6):1040-5.). The associated mechanism of action may be, at least partially, due to an increase in norepinephrine. Catechins are also known to inhibit catechol-O-methyl-transferase (COMT), an enzyme that degrades norepinephrine (Borchardt R T, Huber J A, Catechol O-methyltransferase, 5. Structure-activity relationships for inhibition by flavonoids. J Med Chem, 1975 Jan;18(1):120-2.). EGCG is both a substrate of and, as a result an inhibitor of, COMT (Li C, Allen A, Kwagh J, Doliba N M, Qin W, Najafi H, Collins H W, Matschinsky F M, Stanley C A, Smith T J. Green tea polyphenols modulate insulin secretion by inhibiting glutamate dehydrogenase. J Biol Chem. 2006 Apr 14:281(15:10214-21. Epub 2006 Feb 13). In turn, norepinephrine inhibits degradation of intracellular cyclic AMP (cAMP), an important signaling molecule involved in many metabolic processes including thermogenesis. Furthermore, EGCG has been shown to be an inhibitor of glutamate dehydrogenase, which regulates insulin secretion (Lu H, Meng X, Yang C S. Enzymology of methylation of tea catechins and inhibition of catechol-O-methyltransferase by (-)-epigallocatechin gailate. Drug Metab Dispos. 2003 May:31(5):572-9.).

In an embodiment of the present invention, which is set forth in greater detail in Example 1, the diet supplement may include an N-olyl-phosphatidyl ethanolamine (“NOPE”)/EGCG blend. A serving of the diet supplement may include from about 0.1 mg to about 10 mg of N-olyl-phosphatidyl ethanolamine (NOPR)/EGCG blend. The preferred dosage, in a serving of said diet supplement, comprises about 1 mg of N-olyl-phosphatidyl ethanolamine (NOPE)/EGCG blend.

Russian Tarragon Extract

Russian Tarragon (Artemisia dracunculus) is a perennial herb widely used in cooking. Historically it has been use as a natural blood cleanser and as a treatment for headaches and dizziness. Current studies are examining the use of the ethanolic extract of Russian Tarragon called for the treatment of hyperglycemia associated with diabetes. The toxicology of this extract has been evaluated, and shown to be safe and non-toxic (Ribnicky D M, Poulev A, O'Neal J, Wnorowski G, Malek D E, Jager R, Raskin I. Toxicological evaluation of the ethanolic extract of Artemisia dracunculus L. for use as a dietary supplement and in functional foods. Food Chem Toxicol 2004 Apr;42(4):586-98). Furthermore, it has been demonstrated to reduce blood glucose levels in diabetic mice (Ribnicky D M, Poulev A, Watford M, Cefalu W T, Raskin I. Antihyperglycemic activity of Tarralin™, an ethanolic extract of Artemisia dracunculus L. Phytomedicine. In Press. Corrected Proof, Available online 2 November 2005). Additionally, an essential oil extracted from Artemisia dracunculus may have potential therapeutic effects as an anticonvulsant and mild sedative (Sayyah M, Nadjafnia L, Kamalinejad M. Anticonvulsant activity and chemical composition of Artemisia dracunculus L. essential oil J Ethnopharmacol 2004 Oct.94(2-3):283-7.).

In an embodiment of the present invention, which is set forth in greater detail in Example 1, the diet supplement may include Russian Tarragon Extract (Artemisia dracunculus L. plant). A serving of the diet supplement may include from about 0.1 mg to about 10 mg of Russian Tarragon Extract (Artemisia dracunculus L. plant). The preferred dosage, in a serving of said diet supplement, comprises about 1 mg of Russian Tarragon Extract (Artemisia dracunculus L. plant).

N-acetyl Tyrosine

N-acetyl tyrosine is a stable form of the amino acid tyrosine. Tyrosine is a nonessential amino acid, meaning that it can be synthesized by the body. Amino acids are the building blocks of protein. Tyrosine can also function as a neurotransmitter precursor and is converted into neuronal signaling molecules such as dopamine, norepinephrine, and epinephrine. It is known that increasing levels of neurotransmitter precursors in the brain stimulates further neurotransmitter production (Wurtman R J, Hefti F, Melamed E. Precursor control of neurotransmitter synthesis. Pharmacol Rev. 1980 Dec:32(4)315-35). As such, Tyrosine levels in the brain and the subsequent conversion into neurotransmitter has been shown to increase in response to increased dietary Tryrosine (Fernstrom J D, Fernsstrom M H. Dietary effects on tyrosine availability and catecholamine synthesis in the central nervous system, possible relevance to the control of protein intake. Proc Nutr Soc. 1994 Jul:53(2):419-29.). Conversely. Tyrosine depletion in humans has been shown to have detrimental effects on mood (Leyton M, Young S N, Pihl R O, Elezadi S, Lauze C, Blier P, Baker G B, Benkelfat C. Effects on mood of acute phenylalanine/tyrosine depletion in healthy women. Neuropsychopharmacology 2000 Jan:22(1):52-63.) and cognitive performance (Grevet E H, Tietzmann M R, Shansis F M, Hasteripflugl C, Santana L C, Forster L, Kapczinskil F, Irquierdo I. Behavioural effects of acute phenylalanine and tyrosine depletion in healthy male volunteers. J Psychopharmacol, 2000 Mar;16(1:51-5.).

Tyrosine supplementation has be used to successfully improve mood and cognitive performance during periods of stress (Banderet L E, Liebeman H R Treatment with tyrosine a neurotransmitter precursor, reduces environmental stress in humans. Brain Res Bull 1989 Apr:22(4):759-62). One study has demonstrated a reduction in blood pressure in addition to cognitive improvement (Deijen J B, Oriebeke J F. Effect of tyrosine on cognitive function and blood pressure under stress. Brain Res Bull 1994:33(3):319-23.) via Tyrosine supplementation. Mental performance during fatigue has been shown to improve with increased Tyrosine (Neri D F, Wiegmann D, Stanny R R, Shappell S A, McCardie A, McKay D L. The effects of tyrosine on cognitive performance during extended wakefulness. Aviat Space Environ Med. 1995 Apr:68(4):313-9.) supplementation.

In an embodiment of the present invention, which is set forth in greater detail in Example 1, the diet supplement may include N-acetyl tyrosine. A serving of the diet supplement may include from about 0.1 mg to about 10 mg of N-acetyl tyrosine. The preferred dosage, in a serving of said diet supplement, comprises about 1 mg of N-acetyl tyrosine.

Gymnema Sylvestre

Gymnema Sylvestre is a plant which is used in traditional Eastern medicine as a treatment for diabetes due to its ability to inhibit glucose absorption in addition to its ability suppress the taste of ‘sweetness’.

Gymnema extract has been shown to be an effective diabetes treatment in rats (Okabayashi Y, Tani S, Fujisawa T, Koide M, Hasegawa H, Nakamura T, Fujii M, Otsuki M. Effects of Gymnema sylvestre R.Br on glucose homeostasis in rats Diabetes Res Clin Pract. 1990 May-Jun:9(2):143-8.). This is likely due to the ability of Gymnema to suppress glucose absorption in rat intestine (Shimizu K, Iino A, Nakajima J, Tanaka K, Nakalyo S, Urakawa N, Atsuchi M, Wada T, Yamashita C, Suppression of glucose absorption by some fractions extracted from Gymnema sylvestre leaves J Vet Med Sci. 1997 Apr:59(4):245-51); to lower glucose content in tissue of rats (Chattopadhyay R R. Possible mechanism of antihyperglycemic effect of Gymnema sylvestre leaf extract, part I Gen Pharmacol 1998 Sep;31(3):495-6.); and to suppress neural responses to sweet taste in rats and mice (Imoto T, Miyasaka A, Ishima R, Akasaka K. A novel peptide isolated from the leaves of Gymnema sylvestre-I. Characterization and its suppressive effect on the neural responses to sweet taste stimuli in the rat Comp Biochem Physiol A 1991;100(2):309-14. Ninomiya Y, Imoto T. Gurmarin inhibition of sweet taste responses in mice. Am J Physiol. 1995 Apr;268(4 Pt 2),R1019-25.).

Moreover, Gymnema has further been shown to be effective in treating diabetes in humans by both lowering blood glucose along with increasing insulin levels (Shanmugasundaram E R, Rajeswari G, Baskaran K, Rajesh Kumar B R, Radha Shanmugasundaram K, Kizar Ahmath B. Use of Gymnema sylvestre leaf extract in the control of blood glucose in insulin-dependent diabetes melitus J Ethnopharmacol 1990 Oct;30(3):281-94; Baskaran K, Kizar Ahamath B, Radha Shanmugasundaram K, Shanmugasundaram E R. Antidiabetic effect of a leaf extract from Gymnema sylvestre in non-insulin-dependent diabetes melitus patients. J Ethnopharmacol. 1990 Oct;30(3):295-300.). Gymnema Sylvestre extract can be combined with HCA to produce greater weight loss (Preuss H G, Garis R I, Bramble J D, Bagchi D, Bagchi M, Rao C V, Satyanarayana S. Efficacy of a novel calcium/potassium salt of (-)-hydroxycitric acid in weight control Int J Clin Pharmacol Res 2005;25(3):133-44.) as experimentally evidenced.

In an emodiment of the present invention, which is set forth in greater detail in Example 1, the diet supplement may include Gymnema Sylvestre Leaf Extract. A serving of the diet supplement may include from about 1 mg to about 1000 mg of Gymnema Sylvestre Leaf Extract. The preferred dosage, in a serving of said diet supplement, comprises about 133 mg of Gymnema Sylvestre Leaf Extract.

Withania Somnifera Root Extract

Withania Somnifera (Ashwagandha Winter Cherry) is an herb used in traditional East Indian medicine. Withania Somnifera is reported to have a number of beneficial effects including antioxidant and antistress (Mishra L C, Singh B B, Dagenais S. Scientific basis for the therapeutic use of Withania somnifera (ashwagandha); a review Altern Med Rev. 2000 Aug:5(4):334-46) activities. It is also considered to be an adaptogen and therefore, may possess non-specific protective effects, wherein this has been demonstrated in animal studies (Dhuley J N. Asaptogenic and cardioprotective action of ashwagandha in rats and frogs J Ethnopharmacol. 2000 Apr;70(1):57-63.). In rats, Withania Somnifera has a positive effect on mood by reducing stress and anxiety (Bhattacharya S K, Bhattacharya A, Sairam K, Ghosal S. Anxiolytic-antidepressant activity of Witharia somnifera glycowithanolides, an experimental study. Phytomedicine 2000 Dec;7(6):463-9.). Moreover, Withania Somnifera has been shown to attenuate both age-associated and chemica-induced cellular and tissue oxidative damage in rats (Gupia S K, Dua A, Vohra B P. Withania somnifera (Ashwagandha) attenuates antioxidant defense in aged spinal cord and inhibits copper induced lipid peroxidation and protein oxidative modifications Drug Metabol Drug Interact, 2003:19(3):211-22.).

Human clinical trials examining Withania Somnifera have been conducted. One clinical trial demonstrated potential for Withania Somnifera to treat arthritic (Kuikami R R, Patki P S, Jog V P, Gandage S G, Patwardhan B, Treatment of osteoarthritis with a herbomineral formulation, a double-blind, placebo-controlled, cross-over study J Ethnopharmacol, 1991 May-Jun;33(1-2):91-5).

In an embodiment of the present invention, which is set forth in greater detail in Example 1, the diet supplement may include Withania Somnifera Root Extract. A serving of the diet supplement may include from about 0.1 mg to about 10 mg of Withania Somnifera Root Extract. The preferred dosage, in a serving of said diet supplement, comprising about 1 mg of Withania Somnifera Root Extract.

Calcium and Potassium double salt of Garcinia Cambogis Extract (supplying about 60% Hydroxycitric Acid)

Hydroxycitric acid (HCA) is extracted from the fruit of the Garcinia Cambogia plant, it has been shown to inhibit fat production and suppress appetite and as such is used to control weight by virtue of these characteristics.

U.S. Pat. No. 6,875,891, entitled “Process for Preparing Highly Water Soluble Double Salts of Hydroxycitric Acid Particularly Alkali and Alkaline Earth Metal Double Salts” describes a method for producing highly water-soluble Calcium and Potassium Hydroxycitric Acid salts which are odorless and essentially tasteless. The process involves the steps of precipitating sparingly soluble alkaline earth metal salts of Hydroxycitric acid from an aqueous extract of plants belonging to the Garcinia species, dissolving said alkaline earth metal salts in aqueous alkali and adjusting the pH of said alkaline solution by adding an extract of purified Garcinia fruit extract. From this process, the Calcium salt of Hydroxycitric Acid can be precipitated. Additionally, said Calcium salt can be treated with Potassium Hydroxide to form the Potassium and Calcium double salt of Hydroxycitric Acid which can further be purified by treatment with activated charcoal, filtered and spray dried.

HCA has been shown to inhibit fatty acid synthesis and repress appetite in rats (Watson J A, Fang M, Lowenstein J M. Tricarballylate and hydroxycitrate: substrate and inhibitor of ATP citrate oxaloacetate lyase Arch Biochem Biophys 1969 Dec;135(1):209-17. Louter-van de Haar J, Wielinga P Y, Scheurink A J. Nieuwenhuizen A G. Comparison of the effects of three different (-)-hydroxycitric acid preparations on food intake in rats Nutr Metal (Lond), 2005 Sep 13:2:23.) and is known to be a competitive inhibitor or ATP citrate lyase, an enzyme necessary for the conversion of carbohydrates into fat. By its actions of competitive inhibition of ATP citrate lyase, it reduces that amount of storable fat. Garcinia Cambogia extract can also improve glucose metabolism in mice (Hayamizu K, Hirakawa H, Oikawa D, Nakanishi T, Takagi T, Tachibana T, Furuse M. Effects of Garcinia cambogia extract on serum leptin and insulin in mice, Fitoterapia, 2003 Apr:74(3:267-73).

Several human clinical trials have demonstrated that the HCA extract of Garcinia Cambogia can be used safely and has beneficial effects in terms of weight management, Moreover, HCA has been shown to reduce caloric intake (Westerterp-Plantenga M S, Kovacs E M. The effect of (-)-hydroxycitrate on energy intake and safety in overweight humans Int J Obes Relat Metab Disord. 2002 Jun;26(6):870-2) and increase fat oxidation during exercise in untrained men and women (Lim K, Ryu S, Nho H S, Choi S K, kwon T, Suh H, So J, Tomita K, Okuhara Y, Shigematsu N. (-)-Hydroxycitric acid ingestion increases fat utilization during exercise in untrained women J Nutr Sci Vitaminol (Tokyo) 2003 Jun;49(3):163-7: Tomita K, Okuhara Y, Shigematsu N, Suh H, Lim K (-)-hydroxycitrate ingestion increases fat oxidation during moderate intensity exercise in untrained men. Biosci Biotechnol Biochem, 2003 Sep;67(9):1999-2001.). The results of a randomized controlled trial which combined data from two earlier trials demonstrated that daily HCA and chromium supplementation together with moderate exercise over an 8-week period resulted in increased weight loss as compared to placebo, as well as resulted in an improved blood cholesterol profile (Preuss H G, Garis R I, Bramble J D, Bagchi D, Bagchi M, Rao C V, Satyanarayana S. Efficiency of a novel calcium/potassium salt of (−)-hydroxycitric acid in weight control Int J Clin Pharmacol Res 2005;25(3:133-44.). It was also noted that serotonin levels were significantly increased by HCA. Serotonin is a neurotransmitter which, when low, signals hunger and particularly carbohydrate cravings.

In an embodiment of the present invention, which is set forth in greater detail in Example 1 below, The diet supplement may include the Calcium and Potassium double salt of Garcinia Cambogia Extract standardized to about 60% Hydroxycitric Acid. A serving of the diet supplement may include from about 100 mg to about 5 g of the Calcium and Potassium double salt of Garcinia Cambogia Extract standardized to about 60% Hydroxycitric Acid. The preferred dosage, in a serving of the diet supplement, comprises about 1.555 g of the Calcium and Potassium double salt of Garcinia Cambogia Extract standardized to about 60% Hydroxycitric Acid.

Rhodiola Rosea

Rhodiola Rosea is also known as ‘Golden root’, ‘Arctic root’ and Crenulin. Typically, it is considered to be an ‘adaptogen’ due to the observed ability of Rhodiola to confer increased resistance to multiple stresses, both mental and physical (Kelly G S, Rhodiola rosea: a possible plant adaptogen Altern Med Rev. 2001 Jan:6(3):293-302.). The mechanism of action for Rhodiola Rosea appears to be primarily its ability to increase the levels of monamine neurotransmitters such as serotonin, dopamine and norepinephrine (Stancheva S L, Mosharrof A, Effect of the extract of Rhodiola rosea L, on the content of the brain biogenic monamines, Med Physiol 1987:40:85-87.). In vivo experiments in rats show that Rhodiola possesses both cardioprotective (Lishmanov IuB, Maslova L V, Maslov L N, Dan'shina E N, [The anti-arrhythmia effect of Rhodiola rosea and its possible mechanism] Biull Eksp Biol Med. 1993 Aug:116(8):175-6.) and anticancer properties (Udinlsev S N, Shakhov V P. The role of humoral factors of regenerating liver in the development of experimental tumors and the effect of Rhodiola rosea extract on this process. Neoplasma 1991: 38(3):323-331.). Using in vitro studies on human cells, it was demonstrated that Rhodiola extract has marked protective and antioxidant activity (De Sanctis R, De Bellis R, Scesa C, Mancini U, Cucchianni L, Dacha M, in vitro protective effect of Rhodiola rosea extract against hypochlorous acid-induced oxidative damage in human erythrocytes Biofactors, 2004:20(3):147-59.).

In human clinical trials, Rhodiola has been shown to improve mental performance (Darbinyan V, Kleyan A, Panossian A, Gabriellian E, Wikman G, Wagner H, Rhodiola rosea in stress induced fatigue—a double blind cross-over study of a standardized extract SHR-5 with a repeated low-dose regimen on the mental performance of healthy physicians during night duty. Phytomedicine, 2000 Oct:7(5):365-71.; Shevtsov V A, Zholus B I, Shervarly V I, Vol'skij V B, Korovin Y P, Khristich M P, Roslyakova N A, Wikman G. A randomized trial of two different doses of a SHR-5 Rhodiola rosea extract versus placebo and control of capacity for mental work. Phytomedicine, 2003 Mar:10(2-3):95(105.) and reduce stress-induce fatigue without side-effects (Spasov A A, Wikman G K, Mandrikov V B, Mironova I A, Neumoin W. A double-blind, placebo-controlled pilot study of the stimulating and adaptogenic effect of Rhodiola rosea SHR-5 extract on the fatigue of students caused by stress during an examination period with a repeated low-dose regimen. Phytomedicine, 2000 Arr;7(2):85-9.). Furthermore, Rhodiola can also improve endurance exercise performance (De Beck K, Eijnde B O, Ramaekers M, Hespel P, Acute Rhodiola rosea intake can improve endurance exercise performance, Int J Sport Nutr Exerc Metab, 2004 Jun;14(3):298-307.).

In an embodiment of the present invention, which is set forth in greater detail in Example 1, the diet supplement may include Rhodiola Rosea Leaf Extract. A serving of the diet supplement may include from about 1 mg to about 1 g of Rhodiola Rosea Leaf Extract. The preferred dosage, in a serving of said diet supplement, comprises about 124 mg of Rhodiola Rosea Leaf Extract.

The diet supplement according to this invention provides a method for causing rapid weight loss, controlling appetite, managing stress, supporting relaxation, and supporting mental well-being. Advantageously, consumption of the diet supplement is combined with a reduced calorie diet and a program of regular exercise.

According to various embodiments of the present invention, the diet supplement may be consumed in any form. For instance, the dosage form of the diet supplement may be provided as, e.g., a powder beverage mix, a liquid beverage, a ready-to-eat bar or drink product, a capsule, a tablet, a caplet, or as a dietary gel. The most preferred dosage form is a caplet.

Preferably, the diet supplement is consumed by an individual in accordance with the following method: As a diet supplement, 2 caplets may be taken with an 8 oz. glass of water three times daily. Preferably each serving, comprised of 2 caplets may be consumed approximately 30 to 60 minutes before each meal, e.g., breakfast, lunch and dinner. In this manner, the diet supplement may cause rapid weight loss, control appetite, manage stress, support relaxation, combat fatigue and support mental well-being for an extended period of time, e.g., all day.

Furthermore, the dosage form of the diet supplement may be provided in accordance with customary processing techniques for herbal and dietary supplements in any of the forms mentioned above. Furthermore, the diet supplement set forth in the example embodiments herein may contain any appropriate number and type of excipients, as is well known in the art.

In addition, the present invention relates to a method of manufacturing a diet supplement for causing rapid weight loss, controlling appetite, and managing stress, supporting relaxation, combating fatigue and supporting mental well-being. For example, the method of manufacturing a diet supplement may include the step of mixing one or more of the Calcium and Potassium double salt of Garcinia Cambogia Extract, supplying 60% Hydroxycitric Acid, Gymnema Sylvestre Leaf Extract, Rhodiola Rosea Root Extract, Theanine (gamma-glutamylethylamide), Astaxanthin, Chromium Polynicotinate, Hoodia Gordonii (e.g., an extract that does not contain any extract from the root of the plant), N-olyl-phosphatidyl ethanolamine (NOPE)/EGCG blend, Vinpocetine, Russian Tarragon Extract (Artemisia dracunculus L. PLANT), N-acetyl tyrosine, Withania Somnifera Root Extract, Microcrystalline Cellulose, Dicalcium Phosphate Dihydrate, Stearic Acid, Titanium Dioxide, Water, Propylene Glycol, Sodium Carboxymethylcellulose, Maltodextrin, Dextrose Monohydrate, Soy Lecithin, Polysorbate 80, Croscarmellose Sodium, Magnesium Stearate, Silicon Dioxide, Sucralose, and Ascorbic Acid. The method of manufacturing the diet supplement may also include the step of checking for uniformity/homogeneity. In addition, the method of manufacturing the diet supplement may include the step of aliquoting the mixture into a serving, e.g., for compression into a caplet.

Although the following example illustrates the practice of the present invention in one of its embodiments, the example should not be construed as limiting the scope of the invention. Other embodiments will be apparent to one skilled in the art from consideration of the specification of the following example.

### EXAMPLES

### Example 1

A diet supplement for promoting rapid weight loss, controlling appetite, managing stress, supporting relaxation, combating fatigue and supporting mental well-being is provided, the diet supplement comprising the Calcium and Potassium double salt of Garcinia Cambogia Extract standardized to about 60% Hydroxycitric Acid (1.55500 g), Gymnema Sylvestre Leaf Extract (0.133000 g) standardized to 25% Gymnemic Acids, Rhodiola Rosea Extract (0.124000 G) standardized to 3% Rosavins. Theanine (0.052000 g). Astaxanthin Algae Extract (0.00500 g), Chromium-Polynicotinate (0.000133 g), Hoodii Gordonii (0.020000 g) plant without the roots. N-olyl-phosphatidyl ethanolamine (NOPE)/EGCG blend (0.00100 g) standardized to 23% NOPE, 20% Polyphenols, 14% EGCG, Vinpocetine (0.00100 g). Russian Tarragon Extract (0.00100 g), N-acetyl tyrosine (0.00100 g), and Withania Somnifera Root Extract (0.00100 g) standardized to 1.5% Withanolides.

Directions: As a diet supplement, 2 caplets are administered with an 8 oz. glass of water threee times daily. Preferably, each two caplet serving may be consumed approximately 30 to 60 minutes before each meal, e.g., breakfast, lunch and dinner.

1-20. (canceled)

21. A method for promoting weight loss comprising the oral administration to a mammal a dietary supplement comprising:
Hoodia Gordonii or an extract of Hoodia Gordonii;
a Calcium and Potassium double salt of Garcinia Cambogia Extract supplying 60% Hydroxycitric Acid;
a leaf extract of Gymnema Sylvestre;
Chromium Polynicotinate; and
a blend of N-olyl-phosphatidyl Ethanolamine/EGCG,
wherein the Hoodia Gordonii or Hoodia Gordonii is devoid of material from the roots of said Hoodia Gordonii.

22. The method of claim 21 wherein the dietary supplement further comprises at least one ingredient selected from the group comprising an extract of Russian Tarragon and Vinpocetine.

23. The method of claim 21, wherein the dietary supplement is administered to the mammal at least once daily.

24. A method for promoting weight loss while concomitantly reducing feelings of anxiety and stress in a mammal comprising the oral administration to a mammal a dietary supplement comprising:
Hoodia Gordonii or an extract of Hoodia Gordonii; and
an effective amount of Theanine,
wherein the Hoodia Gordonii or Hoodia Gordonii extract to devoid any material from the roots of said Hoodia Gordonii.

25. The method of claim 24 wherein the dietary supplement further comprises at least one ingredient selected from the group comprising an extract of Rhodiola Rosea, N-acetyl Tyrosine and an extract of Withania Somnifera Root wherein said at least one ingredient is provided in an amount effective to aid in reducing feelings of anxiety and stress in the mammal.

26. The method of claim 24 wherein the dietary supplement further comprises an extract of Russian Tarragon wherein said Russian Tarragon is provided in an amount effective to induce a feeling of relaxation in the mammal.

27. The method of claim 24 wherein the dietary supplement further comprises at least one ingredient selected from the group comprising an extract of Algae providing Astaxanthin, and Vinpocetine wherein said at least one ingredient is provided in an amount effective to support mental well-being in the mammal.

28. The method of claim 24, wherein the dietary supplement is administered to the mammal at least once daily.
